(19) United States
(12) Patent Application Publication
(10) Pub. No.: US 2020/0399710 A1
(43) Pub. Date: Dec. 24, 2020

(54) METHOD OF IDENTIFYING TUMOR SPECIFIC MACROMOLECULAR ISOFORMS

(71) Applicant: Isommune, LLC, Avila Beach, CA (US)

(72) Inventor: Christian Barrett, Pacific Beach, CA (US)

(21) Appl. No.: 16/969,168

(22) PCT Filed: Feb. 7, 2019
(86) PCT No.: PCT/US2019/017013

§ 371 Date: Aug. 11, 2020

Related U.S. Application Data
(60) Provisional application No. 62/629,319, filed on Feb. 12, 2018.

(51) Int. Cl. C12Q 1/6886; C07K 16/30

(52) U.S. Cl. CPC C12Q 1/6886; C07K 16/30

(57) ABSTRACT

The present disclosure provides methods for the identification of tumor-specific isoforms of polymorphic gene expression products. Methods of using said isoforms in the diagnosis and/or treatment of cancers and neoplasias are also provided.

### FIELD

The present disclosure relates generally to the field of macromolecular targets for use in drug discovery and disease diagnosis and, more particularly, cancer diagnosis.

### BACKGROUND

Over the last two decades, numerous academic and industrial efforts have sought to identify tumor-specific genes that would enable targeted anti-cancer therapies. Aside from some in the class of cancer-germline (e.g., cancer/testis) genes, few have been found. In retrospect, the “gene” concept critically hindered these efforts to discover tumor-specific molecules because the word “gene” is a collective term for the aggregate of all isoforms made from a genomic locus and does not refer to any one particular molecule. Malignant and normal tissue types can be distinguished by patterns of differential isoform usage, but when measured in aggregate at the “gene” level the isoform-specific differences are at best recognized as “gene overexpression”.

The distinction between a gene that is overexpressed in tumors relative to normal tissues and an actual tumor specific molecule has major implications for a targeted therapy. Current therapies based on gene targets that are overexpressed, such as all FDA-approved anti-cancer monoclonal antibody therapies, were designed to non-selectively target all isoforms of the target gene. As a consequence of non-selectively targeting all isoforms of target genes, these therapies have severe restrictions on the dose, duration, and extent to which they can be applied and are often accompanied by toxicity side effects. Technology for measuring gene expression is mature and has been used by countless groups worldwide to search for genes that are only expressed in tumors. Few have been reported. Aside from fusion transcripts, on the other hand, the full extent to which tumor-specific mRNA isoforms exist is unknown.

Molecules that reside in the cell membrane of tumor cells and that are exposed on the outer surface represent potential weaknesses that can be exploited to kill tumor cells. Therapeutic monoclonal antibodies, the major anti-cancer drug class, are specially engineered to bind to a specific type of molecule on the surface of tumor cells and kill them. A very recent and exciting development in cancer therapy is the introduction of Chimeric Antigen Receptor T-cells (CAR-T cells), a live cell (“living drug”) that is engineered to seek and kill tumor cells. In essence, these cells are engineered to have a monoclonal antibody that is fixed to their surface and that guides them to tumor cells.

A major limitation of all FDA-approved anti-cancer therapeutic monoclonal antibodies and all CAR-T cell therapies under development is that they target cell surface molecules that are not tumor-specific, but that are also expressed on a range of normal tissues. Consequently, normal organ toxicity and serious side effects are associated with these drugs—severely restricting the dose, duration, and extent to which they can be used to save lives. So, while these therapies are more tumor selective than traditional anti-cancer therapies like chemotherapy, they do not fulfill the ultimate goal of targeted therapy to completely and selectively eradicate all tumor cells of a cancer.

There exists, therefore, an unmet need for tumor-specific cell surface molecules and monoclonal antibody and CAR-T cell therapies based on those tumor-specific molecules. Disclosed herein are methods that transcend the limiting “gene” concept, focusing instead on isoforms specific to tumors. The disclosed methods harness the discriminative potential of such isoforms and serve as the basis for a new generation of highly targeted therapies that avoid the current technologies' severe restrictions on the dose, duration, extent, and toxic side effects.

### SUMMARY

The present disclosure provides methods of making a synthetic polynucleotide, comprising the steps of: selecting a cellular compartment; identifying a first set of genetic sequences expressed in said cellular compartment; identifying a second set of genetic sequences expressed as multiple isoforms; generating a subset of the first and second sets of genetic sequences, said subset comprising sequences that are both (a) expressed in the selected cellular compartment and (b) capable of being expressed in multiple isoforms; and finally, generating polynucleotide primers that are complementary to one or more sequences from the subset of genetic sequences. The primers may further comprise sequences such as sequence tags, bar codes, etc., that provide for the use of universal sequencing primers and/or unique genetic identifiers.

In some embodiments, the methods described herein may then comprise amplifying one or more genetic sequences of the previously identified subset of genetic sequences, especially where those sequences are identified as being uniquely expressed in, or significantly overexpressed in, tumor tissue vs. normal tissue, or vice versa.

The methods described herein may further comprise steps including identifying the protein products of the previously identified subset of genetic sequences in tumor and/or normal tissue, and isolating the protein products of said subset of genetic sequences from tumor tissue.

The polynucleotide primers used in the methods of the present disclosure may further comprise additional sequences outside of the region containing the sequence of the identified isoforms, and may further comprise universal sequencing sites, sequences for the identification of each primer and/or sequences for use in the amplification of isoforms identified using said primers.

Also described among the embodiments of the present disclosure are methods of obtaining one or more tumor-specific biomarkers, comprising the steps of: selecting a cellular compartment; identifying a first set of genetic sequences expressed in said cellular compartment; identifying a second set of genetic sequences expressed as multiple isoforms; and generating a subset of said first and second sets of genetic sequences, said subset comprising one or more genetic sequences known to be both (a) expressed in said cellular compartment and (b) capable of being expressed in multiple isoforms; and finally, extracting, purifying, or expressing the product of one or more genetic sequences from said subset of genetic sequences.

In some embodiments, these methods may further comprise amplifying one or more genetic sequences of the subset of genetic sequences that are identified as being uniquely expressed in, or significantly overexpressed in, tumor tissue vs. normal tissue, or vice versa.

As above, these methods may additionally include identifying the protein products of the identified tumor-specific isoforms, or isoforms known or found to be significantly enriched in tumor tissue as opposed to normal tissue. The methods described herein may further comprise isolating the protein products of one or more of the genetic sequences thereby identified, or producing said protein products by heterologous expression. The polynucleotide primers used in these methods may further comprise additional sequences outside of the region containing the sequence of the identified isoforms, and may further comprise universal sequencing sites, sequences for the identification of each primer and/or sequences for use in the amplification of isoforms identified using said primers.

In some embodiments, the methods as described herein include generating an antibody against the protein product or products of the isoforms identified according to the present disclosure. Antibody generation may comprise immunizing an animal with said protein product or protein isoform, followed by extraction of immunoglobulins from the animal. The animal may be a mammal or bird, such as a human, mouse, rat, guinea pig, dog, cat, pig, goat, horse, donkey, mule, domestic cow, llama, alpaca, guanaco, dromedary, Bactrian camel, other camel, chicken, duck, turkey, goose, peafowl or pigeon.

According to the methods described herein, immunoglobulins and/or B cells with specificity to said one or more products of said genetic sequences may be purified, providing for the generation of polyclonal as well as monoclonal antibodies. Antibodies according to the present disclosure may be generated by in vitro or ex vivo means. For example, any part of said antibody may generated by phage display.

In some embodiments, the present disclosure describes methods of diagnosing cancer, comprising: obtaining a sample from a subject, said sample containing one or more proteins or nucleic acids; and identifying and/or quantifying the presence of one or more tumor-specific biomarkers; wherein said tumor-specific biomarkers are obtainable and/or detectable according to the methods described above.

### DETAILED DESCRIPTION

The number of protein and RNA isoforms made from the human genome is vast, encompassing >500,000 known isoforms. Provided herein are methods of identifying isoforms, in such a manner as to render them suitable for use as diagnostic markers, prognostic markers, or drug targets, especially targets of antibodies (including minibodies, single-chain variable fragments (ScFvs), diabodies, bispecific or multispecific antibodies, and the like), T-cell receptors, synthetic T-cell receptors, selective binding peptides, ligand-based targeting moieties and the like, and any combination thereof.

Certain of the methods of the present disclosure provide for identifying isoforms of interest, and precisely specifying the experiments needed to validate the target (i.e. to confirm that isoform A is actually on the cell surface in tumor types X, Y, and Z.) If a candidate target is validated, then it can be staged for pre-clinical development. If a candidate target is not validated, then it can be dropped from further consideration. Either way, lengthy open-ended investigations are avoided; hypotheses are systematically confirmed, one-by-one in an ongoing fashion, to efficiently reveal therapeutically and commercially valuable molecules. Together, then, the discovery technology and its deployment strategy is an efficient approach for systematically discovering the tumor-specific isoforms that exist.

In some embodiments of the methods and the compositions of the present disclosure, a method is provided for the identification of one or more protein, nucleic acid, or genetic isoforms that are associated with, causative of, diagnostic of, or prognostic of, one or more cancerous, precancerous, or neoplastic conditions. In some embodiments, said protein, nucleic acid, or genetic isoforms have been identified from within a collection or database describing protein, nucleic acid, or genetic isoforms as are known in the art. In some embodiments, said protein, nucleic acid, or genetic isoforms are identified de novo by sequencing or characterization of clinical, population, or experimental samples. In some embodiments, said protein, nucleic acid, or genetic isoforms are identified as being associated, involved with, or causative of, one or more cancerous, precancerous, or neoplastic conditions. In some embodiments, said protein, nucleic acid, or genetic isoforms are identified as being associated with, involved with, or causative of, one or more tumors or tumor tissues. In some embodiments, said protein, nucleic acid, or genetic isoforms are identified as being associated with, involved with, or causative of, one or more clonally expanded or neoplastic cell lines within a subject.

As used herein, the term “polymorphic product” refers to one or more product of transcription, translation, recombination, and/or splicing of a DNA sequence, wherein said DNA sequence may give rise to more than one DNA, RNA, or polypeptide product. The basis of polymorphism may be through recombination of an underlying DNA or RNA sequence, through alternative promoter usage, through usage of alternative start codons, though usage of alternative stop codons, through alternative splicing of said sequence, through alternative modification, such as by methylation, acetylation, glycosylation, ubiquitination, or other such covalent modifications, of the resulting RNA or polypeptide product, or through heretofore unrecognized processes. Polymorphism may also reflect genetic deletions, translocations, or mutations affecting splice junctions, start codons, intein junctions and/ or intein sequences, stop codons or any such variation as may result in the production of a more than one gene product from a given transcriptional initiation region within a cell, chromosome, nucleus, or individual organism; or within a population of cells, chromosomes, nuclei, or individual organisms, or other heretofore unrecognized processes.

“Subject” as used herein, has its ordinary and customary meaning in the field as would be understood by one of skill in the art, in view of this disclosure. This term refers to a human or a nonhuman animal, for example selected or identified for a diagnosis, treatment, inhibition, amelioration of a disease, disorder, condition, or symptom. “Subject suspected of having” has its customary and ordinary meaning as understood by one of skill in the art in view of this disclosure. It refers to a subject exhibiting one or more indicators of a disease or condition. In certain embodiments, the disease or condition may comprise one or more of a disease, disorder, condition, or symptom.

“Administering” has its ordinary and customary meaning in the field as would be understood by one of skill in the art, in view of this disclosure. This term refers to the act of providing a substance, for example a pharmaceutical agent, dietary supplement, or composition, to a subject, and includes, but is not limited to, administering by a medical professional and self-administration. Administration of the compounds disclosed herein or the pharmaceutically acceptable salts thereof can be via any of the accepted modes of administration for agents that serve similar utilities such as are consistent with the formulation of said compounds. Oral administrations are customary in administering the compositions that are the subject of the preferred embodiments. In some embodiments, administration of the compounds may occur outside the body, for example, by apheresis or dialysis.

In some embodiments, the methods of the present disclosure contemplate the administration of one or more compositions useful for the amelioration or treatment of one or more disorders, diseases, conditions, or symptoms.

Standard pharmaceutical and/or dietary supplement formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical and/or dietary supplement compositions comprising, consisting of, or consisting essentially of: (a) a safe and therapeutically effective amount of one or more compounds described herein, or pharmaceutically acceptable salts thereof; and (b) a pharmaceutically acceptable carrier, diluent, excipient or combination thereof.

The terms “pharmaceutically acceptable carrier” and “pharmaceutically acceptable excipient” have their ordinary and customary meanings in the field, as would be understood by one of skill in the art, in view of this disclosure. These terms include any and all appropriate solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations of the compounds disclosed herein. The use of such media and agents for pharmaceutically active substances is well known in the art. Except insofar as any conventional media or agent is incompatible with the active ingredient, the use of these terms in connection with a therapeutic composition is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press. The choice of a pharmaceutically-acceptable carrier to be used in conjunction with the one or more compounds for administration as described herein can be determined by the way the compound is to be administered.

In some embodiments, the methods of the present disclosure contemplate the development of compositions which may be formulated for topical or localized administration. In some embodiments, the methods of the present disclosure further contemplate systemically or parenterally, such as subcutaneously, intraperitoneally, intravenously, intraarterially, orally, enterically, subdermally, transdermally, sublingually, transbuccally, rectally, or vaginally.

Current approaches for discovering DNA mutation neoantigens that can be used as immunotherapeutic molecules have five main steps. First, DNA is isolated from tumor and from unaffected cells of a patient. Second, the exonic regions of the genome are sequenced deeply in both the tumor and normal samples. Third, the sequencing data is analyzed to identify DNA mutations that only occur in the tumor sample. Fourth, each mutation is computationally assessed to determine whether it potentially creates an immunogenic peptide. And fifth, candidate immunogenic peptides are experimentally screened to determine whether they generate an immune response. Those that do become candidate immunotherapeutic molecules in a personalized therapy for the patient. Because such candidate immunotherapeutic molecules are the result of unpredictable mutations that a tumor presents, the DNA-based approach is “reactive” to somatic mutations found in patient's tumor.

Described herein are methods for the identification of neoantigens arising because of, for example, mis-splicing, alternative splicing, alteration of start and/or stop codons, alternative start and/or stop codons and/or tumor-specific rearrangement in tumor cells any or all of which may be the result of genomic rearrangements or alterations to splicing of transcription products within tumor cells. Such neoantigens have distinct potential advantages over neoantigens that are the product of somatic mutations. In distinction, the approach described herein is “proactive” because it systematically seeks candidate immunotherapeutic neoantigens that have been pre-identified and prioritized through complete genome analysis. One or more candidate neoantigen is prioritized based on possessing key immunogenic properties, of being tumor specific, and of occurring in a large fraction of tumors. To prioritize neoantigens based on possessing properties of demonstrated importance for immunogenicity, long contiguous amino acid sequences have been identified, containing multiple overlapping peptides that are predicted to bind multiple MHC-I and MHC-II alleles with both high affinity and stability and induce a Type 1 immune response from both CD4+ and CD8+ T-cells. In some embodiments, peptides may be prioritized based on their likelihood of being processed, and of not outwardly-presenting (to the TCR) peripheral tolerizing pentapeptides that exist in the genomes of microbes. Neoantigens identified by these criteria are useful, for example, as vaccine components. In other embodiments, neoantigens comprising longer peptides can be prioritized, such peptides having the potential for use as targets for the development of diagnostic and/or therapeutic antibodies, SCFv's, chimeric T-cell receptors, and the like, including antisera, polyclonal antibodies, monoclonal antibodies, diabodies, bispecific and/or multispecific antibodies, antibody conjugates, and CAR-T cells. Prioritization criteria may additionally include length, projected stability, presence or absence of repeated sequences, cysteine content, secretion signals, extracellular domains, or the like, or other indicia as are known in the art to affect the suitability of a sequence for antibody development. In still further embodiments, said neoantigens may comprise tumor-specific RNA molecules, such as, for example, messenger RNA, noncoding RNA, long non-coding RNA, or the like, which may, in some embodiments, serve as a target for antisense, RNAi, ribozyme, or CRISPR-based inhibitory and/or diagnostic approaches, or for aptamer-based or oligonucleotide-based targeting of therapeutic and/or diagnostic moieties to tumor cells.

To prioritize isoforms based on tumor-specific expression and on being expressed in a large fraction of tumors, custom algorithms are applied to data sets of ˜36,000 RNA-seq data sets. By prioritizing neoantigens based on computational analyses and RNA-level isolation, significant improvements are made over existing workflows, which require the production of gene products and antibodies, aptamers, etc. early on, a significant and often prohibitive input of labor and materials. The proactive approach described herein allows the generation of antibodies, aptamers, or other such diagnostic or therapeutic molecules to be reserved until the end of the workflow, after validation of the target has already substantially occurred.

In some embodiments, a database is constructed encompassing genes or gene fragments that produce polymorphic gene products, wherein said polymorphic products comprise multiple RNA and/or protein isoforms. In some embodiments, the polymorphic product is an RNA molecule. In some embodiments, the polymorphic product is a messenger RNA (mRNA). In some embodiments, the polymorphic product is a transfer RNA (tRNA). In some embodiments, the polymorphic product is a ribosomal RNA (rRNA). In some embodiments, the polymorphic product is a small inhibitory RNA (siRNA). In some embodiments, the polymorphic product is a noncoding RNA (ncRNA). In some embodiments, the polymorphic product is a long noncoding RNA (lncRNA). In some embodiments, the polymorphic product is one or more other types of RNA. In some embodiments, the polymorphic product is a polypeptide. In some embodiments, the polymorphic product is a protein. In some embodiments, the polymorphic product is assayed to determine which specific isoforms are present. In some further embodiments, tumor tissues are assayed in order to determine the presence of specific isoforms in tumor tissues. In certain embodiments, normal tissues representing the tissues from which said tumors or neoplastic cells arose are assayed to determine the presence or absence of said isoforms. In certain embodiments, isoforms that are present in tumor tissue, but absent in the corresponding normal tissue are selected for further development. In certain embodiments, the tumor-specific isoforms are utilized to generate antibodies, binding peptides, aptamers, nucleotide primers, or ligands that detect elements specific to said isoforms. Elements that may determine or define an isoform include but are not limited to unique splice junctions derived from alternative splicing of the parent gene, the presence or absence 5′RNA regions and/or N-terminal peptide regions that vary due to alternative start codons, and/or the presence or absence of 3′RNA regions and/or C-terminal peptide regions that vary due to alternative stop codons, and the novel transcription and/or gene expression products resulting therefrom. Isoforms may also be derived from, for example, alternative post-translational modifications due to the presence or absence of sites for glycosylation, methylation, acetylation, ubiquitination, or the like, or from any combination of the foregoing bases for isoform specificity, or for other alternative expression types or post-translational modifications as are known in the art and are currently detectable using methods known in the art.

In some embodiments, said antibodies, binding peptides, aptamers, oligonucleotide primers, or ligands that detect elements specific to said isoforms may be utilized to determine the presence, extent, or severity of a tumor, cancer, or neoplastic condition in one or more subjects. In some embodiments, said nucleotide primers, aptamers, or ligands, may comprise one or more of a nucleic acid, a deoxyribonucleic acid (DNA), a ribonucleic acid (RNA), a locked nucleic acid (LNA), a peptide nucleic acid (PNA) or other such nucleic acid derivatives or mimetics as are known in the art. Nucleic acid molecules as described herein may be modified, such as by the attachment of biotin, fluorescein or fluorescein derivatives, by 2′-O-methylation, by 2′-fluorination, or by other means as are known in the art to enhance stability and/or detection of said nucleic acid.

### Target Selection and Validation.

In some embodiments, a database of known genetic sequences having polymorphic expression products is compiled. Such a database can be compiled, for example, by merging existing datatbases such as AceView, GENCODE, H-Inv, RefSeq, RNAcentral, SIB, UCSC, FANTOM5, or other genetic sequence databases, or any combination thereof. Such a database could also be compiled de novo. Each genetic sequence is further identified by the tissues and/or cellular compartments in which it is or may be expressed. Such identification may be by analysis of sequence determinants known to be specific for proteins, peptides, or nucleic acids resident in specific cellular locations or compartments, or by application of prior knowledge of the location of said proteins, peptides, or nucleic acids, or by other methods as are known in the art of sequence analysis or cellular biology, or by combinations thereof. In some embodiments, genetic sequences are prioritized by other criteria, such as their known participation in one or more known cancer, cell proliferation, or other disease pathways. In some embodiments, genetic sequences are prioritized by their known or suspected presence in one or more accessible cellular compartments, on the cell surface, or secreted into the intracellular space.

### Primer Design

In some embodiments, for each locus that is determined to be of interest, isoforms at the locus are identified that can be uniquely distinguished by the nucleotide sequence of a cDNA PCR product that is 50-1000 bp, 75-1250 bp, 1000-1500 bp, 1250-1750 bp, 1500-2500 bp, 100-750 bp, 150-700 bp, 200-800 bp, or 300-500 bp in length. In some embodiments, isoforms at a locus can be uniquely distinguished by the nucleotide sequence of a cDNA PCR product that is greater than 2500 bp in length. In some embodiments, isoforms at a locus can be uniquely distinguished by the nucleotide sequence of a cDNA PCR product that is less than 50 bp in length. In some embodiments, forward, reverse, and/or forward and reverse primer(s) are generated such that said product may be isolated or amplified. In some embodiments, for each possible forward or reverse primer 5′ start position in the locus, a determination is made as to which isoforms that a primer at said position would prime in either the forward or reverse direction. In some further embodiments, said forward or reverse 5′ start positions are grouped so as to constitute an “equivalent primer region.” Said equivalent primer regions may be further evaluated to identify primer pairs that identify unique isoforms. Such unique isoforms may include, for example, sequences that are subject to alternative splicing, alternative transcriptional starts, or known translocations, insertions, or deletions. Primer pairs thus derived are considered to be “equivalent primer region pairs.” For each equivalent primer region pair, actual nucleotide sequences are obtained and/or provided for each equivalent primer region, so as to provide a complementary sequence capable of annealing with, or, alternatively, recapitulating the sequence of, said equivalent primer region. In some embodiments, actual primer sequences will comprise a subsequence of the equivalent primer region. In some embodiments, said primers are further selected based on sequence analyses tools as are known to those of skill in the art to avoid self-annealing, off-target annealing, or annealing in such a manner as to be thermodynamically unfavorable for downstream PCR, sequencing, or other such reactions. Primer pairs thus identified may be utilized as default primer pairs delimiting or indicating a given isoform.

In some embodiments, primer pools are designed such that the set of primers selected is sufficient to measure all, substantially all, most, or a plurality of isoforms at a genomic locus. A primer pool design is comprised of a set of equivalent primer region pairs and the single primer pair to use for each equivalent primer region pair, such that, preferably:
a) a maximum number of isoform-distinguishing products are target amplified in each experiment; and/or
b) no pair of primers have (unwanted) stable thermodynamic interactions; and/or
c) no pair of primers interact such that they produce unwanted amplification products.

In some embodiments, in order to measure all isoforms at a genomic locus, multiple separate primer pools are used. In some embodiments, the multiple primer pools are used in separate experiments. In some embodiments, the multiple primer pools are used in a single experiment. In some embodiments, a single primer pool is used. In some embodiments primer pools are combined such that isoforms at different genomic loci can be assessed. Where primer pools are combined, the primers are compared to one another using sequence analysis tools and methods as are known in the art such that unwanted stable thermodynamic interactions between primers from different genomic locus pools are minimized; and further, said primers are selected such that no pair of primers from different genomic locus pools interact to produce unwanted amplification products. In some embodiments, such comparisons may be made using computational optimization tools. In some embodiments, combinations of primer pools can be obtained which identify all, substantially all, most, or a plurality of isoforms at one genomic locus, at more than 1 genomic locus, at more than 12 genomic loci, at more than 24 genomic loci, at more than 96 genomic loci, at more than 128 genomic loci, at more than 284 genomic loci, at more than 384 genomic loci, at more than 10 genomic loci, or at more than 1536 genomic loci.

In some embodiments, the isolation and/or amplification is by PCR, RT-PCR or qPCR. In some embodiments, the isolation and/or amplification may comprise a direct sequencing step.

In some embodiments, polynucleotide primers are developed that are capable of annealing to known isoforms of the selected and/or prioritized genetic sequences. In some embodiments, the primers may incorporate an additional sequence tag which may allow for downstream identification and/or sequencing of amplification products. In some embodiments, the primers may be utilized to amplify polymorphic RNA sequences. In some embodiments, said primers are utilized to amplify polymorphic RNA sequences from tumor tissues. In some embodiments, the primers are utilized to amplify polymorphic RNA sequences from normal tissues. In some embodiments, the primers are utilized to amplify polymorphic RNA sequences from tumor tissues and from corresponding normal tissue of the same type as that from which the tumor arose. In some embodiments, the polymorphic RNA sequences from said tumor tissue are compared to said polymorphic RNA sequences from said normal tissue, thereby indicating RNA isoforms that are present in, unique to, or overrepresented in, tumor tissue relative to corresponding normal tissue.

Amplification may occur by polymerase chain reaction (PCR), Reverse-Transcription PCR (RT-PCR), real-time RT-PCR, RNA sequencing (RNA-SEQ), sequencing of cDNA, or by any other means as are known in the art. Primers are designed to avoid non-target genomic and/or RNA sequences, to ensure binding to target sequences, and to minimize or eliminate cross-hybridization with non-target molecules. In some embodiments, a software algorithm is provided to analyze said primers in order to avoid non-target genomic and/or RNA sequences, to ensure binding to target sequences, and to minimize or eliminate cross-hybridization with non-target molecules. In some embodiments, primers are designed to amplify all isoforms of interest at any given genetic locus.

### Screening Preparation

Polymorphic RNA sequences that are present in, unique to, or overrepresented in, tumor tissue relative to corresponding normal tissue may be further characterized by sequencing. In some embodiments, said sequences are evaluated to determine whether they represent an mRNA or a separate type of RNA. For RNA isoforms that are present in, unique to, or overrepresented in, tumor tissue relative to corresponding normal tissue, including but not limited to, mRNAs, ncRNAs, tRNAs, lncRNAs, snRNAs, siRNAs, and the like, primers are developed for the detection of said RNA isoforms. In some embodiments, said primers further incorporate additional sequences that may be used for the identification and/or sequencing of the products of any downstream reactions, such as Unique Molecular Identifiers (UMIs) of 3, 4, 5, 6, 7, 8, 9, 10, 11, or 12 bp. UMIs are preferably attached to the 5′ end of the primer, and may further be used for identifying and counting unique priming events. Said primers may further incorporate adapter sequences to enable or facilitate sequencing of any downstream products incorporating said primers. Said primers may also be linked to one or more detectable moieties allowing for the detection of said primer and of molecules annealed thereto. Said detectable moieties may include one or more of a florescent label (such as fluorescein or fluorescein isothiocyanate; additional exemplary fluorescent moieties may be found in The Molecular Probes Handbook, 11^{th} Edition (2010) which is incorporated by reference with regard to its disclosure of fluorescent reagents for the detection and labeling of nucleic acids and proteins), a radiolabel (such as, for example, ¹⁴C, or ³H), a spin label, a colorimetric label, an enzyme, a peptide or protein, a nanoparticle, a liposome, a bead, and/or a micelle or any combination thereof.

For RNA isoforms that are present in, unique to, or overrepresented in, tumor tissue relative to corresponding normal tissue, wherein said RNA isoform comprises one or more mRNAs, said mRNAs may be utilized in heterologous expression systems for the production of the protein isoforms encoded thereby. In some embodiments said mRNAs may be reverse transcribed, and the resulting complementary DNA (cDNA) inserted into an appropriate expression construct, such as a plasmid, viral vector, yeast artificial chromosome, or bacterial artificial chromosome. Expression constructs are well known in the art, and said constructs and methods of their use in the production of recombinant proteins are well known. See, e.g., Green, M. R., and Sambrook, J., Molecular Cloning: A Laboratory Manual, 4^{th} Ed., Cold Spring Harbor Laboratory Press, 2012, which is hereby incorporated by reference with respect to its disclosure of methods of incorporating DNA and/or RNA sequences into heterologous expression systems. Said expression construct may then be transformed or transfected into one or more host cells to effect the expression of the protein product of said cDNA. Exemplary expression systems include but are not limited to bacterial systems (such as E. coli, L. lactis, or V-Max™), fungal systems (such as S. cerevisiae or Schizosaccharomyces pombe), insect cells (such as Drosophila S2 cells) or cultured mammalian cells. Upon expression of said protein isoforms, said protein isoforms may be extracted from said heterologous expression system as appropriate, for example by collection and lysis of recombinant cells or by collection of cell medium or supernatants. Said protein isoforms may then be purified using such methods as are known in the art, including but not limited to affinity chromatography, high-performance liquid chromatography (HPLC), reverse-phase liquid chromatography (RPLC), ion-exchange chromatography, or other such methods are known in the art. Exemplary purification methods are described in Burgess, R. and Deutscher, M., Meth. Enzymol. 436, 2^{nd} Ed., 2009, which is hereby incorporated by reference with respect to its disclosure of methods, systems, techniques, and reagents for the isolation and purification of proteins.

Antibodies, binding peptides, aptamers, nucleotide primers, or ligands that detect elements specific to isoforms may be generated by those means known in the art for the development of such items. In some embodiments, antibodies are generated by immunizing one or more vertebrate animals, such as a mammal or a bird, with the neoantigen of interest in such amounts and in such formulations as to raise an immune response. Exemplary mammals or birds include one or more of a human, mouse, rat, guinea pig, dog, cat, pig, goat, horse, donkey, mule, domestic cow, llama, alpaca, guanaco, dromedary, Bactrian camel, other camel, chicken, duck, turkey, goose, peafowl or pigeon. Once generated, antibodies may be purified by such methods as are known in the art, such as, for example, by affinity purification. Extraction of antibody-generating cells and the development of monoclonal antibodies is also contemplated. In some embodiments, said antibodies are attached to one or more detectable moieties, including but not limited to a fluorescent label, a spin label, a magnetic resonance contrast agent, a radiopaque label, a bead, a colorimetric label, a Raman-active label, a nucleic acid, or other such labels as are known in the art for the detection of antibody binding either in vivo or ex vivo. In some embodiments, said antibodies are further be used as the basis for the generation of diabodies, multivalent antibodies, chimeric T-cell antigen receptors, or the like. In some embodiments, said antibodies are fused, conjugated, or associated with one or more therapeutic moieties, such as, for example, a chemotherapeutic agent. In some embodiments, administration of said one or more antibodies to a subject is followed by the detection of said antibody within a subject, thereby determining the extent and severity of one or more tumors or neoplastic cell groupings within said subject. In some embodiments, administration of said one or more antibodies to a subject results in the treatment, reduction, amelioration, or cure of said tumor or neoplastic cell grouping within said subject.

For example, the methods and compositions as disclosed herein may be used to develop targeted immunotherapies based on neoantigens that commonly arise in HPV-HNSCC tumors because of aberrant RNA splicing. In some embodiments, these missplicing neoantigens are not patient specific, but are relatively common among HPV-HNSCC tumors. Clinically, these missplicing neoantigens will enable peptide vaccine and/or adoptive cell transfer therapies. In some embodiments, said neoantigens are used to develop peptide vaccines and/or adoptive cell transfer therapies. A small fraction of the missplicing neoantigens can be expected to be extracellular epitopes on plasma membrane proteins that could be therapeutic monoclonal antibody targets. In some embodiments, said neoantigens are used to develop therapeutic antibody targets. In some further embodiments, said therapeutic antibodies derived therefrom may comprise one or more of monoclonal antibodies, ScFvs, chimeric T cell receptors, diabodies, bispecific antibodies, antigen binding Fv regions, or other antibodies or antibody-derived molecules as described elsewhere herein. In some embodiments, the methods as described herein allow the identification of candidate immunogenic and tumor-specific missplicing neoantigens by validating the tumor-specific expression of the RNA isoforms that encode them. In some further embodiments, the identified missplicing neoantigens are used in a subject to evoke an appropriate immune response.

Relevant aspects of the methods and compositions of the present disclosure are further illustrated by the following example:

### EXAMPLE 1

Tumor-specific isoforms specific to Head and Neck Squamous Cell Carcinoma (HNSCC) are identified and isolated as follows.

### 1) Isolation of RNA and DNA From 48 HPV-HNSCCs Representing the Tumor Stage, Sex, and Worldwide Geographical Prevalence of the Disease.

Representative tumor samples are obtained and assayed for HPV infection status. HPV-negative samples are retained for analysis. Fresh frozen HNSCC samples are obtained (each at least 100 mg) from multiple sources (such as Cureline, DxBiosamples, ProteoGenex, UC San Diego Moores Cancer Center Biorepository). Twelve each of the samples derive from the United States, India, China, and Europe. In each set of 12, 4 are of each tumor stage I, II, and III. Of the 4 samples of a tumor stage, 2 are from females and 2 are from males. Since tumors originating from the oropharynx have a high likelihood of being HPV+ and the focus of the present study is on HPV-HNSCC, samples from the oropharynx subsite are excluded.

### 2) Isolation of RNA and DNA.

Frozen tissue samples are permeated overnight at −20° C. with RNAlater®-ICE Frozen Tissue Transition Solution (ThermoFisher) to protect RNA integrity during processing. Total RNA and genomic DNA are extracted from a portion of each homogenized tissue lysate (corresponding to 10-30 mg of tissue) using Allprep DNA/RNA Mini Kits (Qiagen) and the lysate remainder archived at −70° C. At least 20 ug of RNA and of DNA are isolated for each case. DNA and RNA quantity (A260) and quality (A260/280 and A260/230) are determined by NanoDrop. Integrity of DNA (Genomic DNA ScreenTape) and RNA (RIN and DV200 are confirmed by Agilent Bioanalyzer and the absence of PCR inhibitors are assessed by qPCR of beta-globin DNA or by RT-qPCR of 18S rRNA.

### 3) Typing of HPV Infection Status.

HPV E6 nested multiplex PCR is used to detect HPV DNA. E6/E7 RNA can sometimes be detected in tumors when HPV DNA is not, so additional RT-qPCR are performed to detect HPV E6/E7 transcripts. As positive controls for the PCR and RT-qPCR experiments, 2A3 (HPV16+) and HeLa (HPV18+) cell lines from American Type Culture Collection (ATCC) are used. The FaDu (HPV-) cell line, also from ATCC, is used as a negative control. All experiments include water as a no-template control.

### 4) Identification of a Large Set of Candidate HPV-HNSCC-Specific RNA Isoforms Encoding Missplicing Neoantigens.

The computational as described in this example has been used pipeline to analyze ˜36,000 tumor and normal tissue RNA-seq data sets and rank-prioritized RNA isoforms by their likelihood of 1) being specifically expressed in HPV-HNSCC tumors and of 2) encoding immunogenic missplicing neoantigens. As a next step, the custom RT-qPCR workflow described herein is used to validate the tumor-specific expression of the top rank isoforms.

### 5) Number of Isoforms to be Screened and Expected Number of Discovered Candidate Isoforms.

Approximately 5,200 top-rank isoforms, encoding missplicing neoantigens for tumor-specific expression, are screened. Based on previous experience of a 0.9% validation rate of tumor-specific expression, it is expected that approximately 5,200×0.009=47 of these isoforms are exclusively expressed in tumors. Following this, the ability of the ˜47 missplicing neoantigens to evoke an appropriate immune response is evaluated. Previous studies collectively indicate a success rate of 5-30%. Thus, of the ·47 missplicing neoantigens identified herein, between 3 and 14 eventually prove suitable for clinical development.

### 6) Dry Lab and Wet Lab Procedures.

Computational pipeline for RNA-seq. The standard RNA-seq computational pipeline for organisms with a sequenced genome has three main components: 1) alignment of RNA-seq “reads” to a reference genome; 2) an isoform model database; and 3) an integration algorithm, whose input is the isoform model database and read alignments and whose output is the expression level of the supplied isoforms. A pipeline that is distinguished from other approaches by its novel methodologies and custom algorithms in each of these components has been developed. One major distinguishing feature of this approach for read alignments is the use of maximally sensitive alignment parameterizations coupled with a custom nucleotide-resolution read-to-isoform correspondence verification algorithm. With this feature, it is possible to maximize the isoform identification information in each RNA-seq data set. A second major distinguishing feature is a custom isoform model database that that has been created by merging all major worldwide isoform model databases.

Candidate tumor-specific, neoantigens-encoding RNA isoforms. To identify RNA isoforms that are pervasively and exclusively expressed in tumors, TCGA RNA-seq data to first identify RNA isoforms expressed in 50-100% of tumors. GTEx and SRA RNA-seq data were used to assess the prevalence and level of these isoforms' expression in normal tissue. The isoforms were then ranked according to their likelihood of being tumor-specific. To identify tumor-specific isoforms that encode tumor-specific neoantigens, those isoforms that did not contain a potential neoantigen or whose potential neoantigen was also encoded by an isoform with normal tissue expression were removed from the ranked list.

Isoform-specific PCR primer design. An RT-qPCR experiment to validate the presence of a particular RNA isoform requires PCR primers that will only amplify a product from that particular isoform. Because there is no preexisting software package that could design primers specific to any arbitrary target RNA isoform in the human genome, a custom software suite was developed to implement the primer design requirements as described herein. Briefly, for a target RNA isoform, the software first identifies sequence signature(s) that are unique to the target isoform. It then formulates an exhaustive set of search parameterizations for primers that allow it to search for primer pairs spanning and/or flanking the signatures in a tractable amount of time. Finally, thermodynamic models are used to confirm that 1) the PCR primers will not amplify genomic DNA or any other product in the human transcriptome and 2) the PCR product will yield a single peak in a melt curve analysis. To ensure adequate coverage of 5,200 candidate tumor-specific RNA isoforms, PCR primers for 9,500 candidate isoforms are initially designed. Such a large initial candidate set is necessary because it has been determined that only ˜55% of isoform-specific PCR primer design attempts are successful due to reasons related to Tₘ requirements, forward and reverse primer compatibility, primer or amplicon sequence length constraints, and amplification of unwanted products.

A custom infrastructure for high-throughput RT-qPCR. Primer oligonucleotides are obtained in 96-well plate format (IDT) and plated into a 384-well format at 300 nM with template cDNA at a concentration of 10 ng/uL into a total reaction volume of 10 uL. qPCR is run for 35 cycles and a standard melt curve is performed. The raw amplification and melt data for a PCR run are exported into a single text file for input into custom software that has been developed to provide for quality control, analysis, and expression quantification. The MIQE guidelines for relative quantification have been implemented in this custom software. Most important was the inclusion of qPCR reaction efficiencies in the quantification calculations. To calculate the efficiency of 384 PCR wells without performing 384 dilution curve experiments, derivative analysis is applied and a linear regression method is used to compute PCR reaction efficiencies. Relative quantification is then performed using three references. Melt curve analysis has been found to be critical for isoform-level investigations, so careful melt curve analysis is used to confirm that the qPCR reaction specifically amplified the intended isoform.

### 7) Initial Validation.

With PCR primers for 5,200 RNA isoforms, tumor-specific isoform expression is confirmed in a two-pass strategy. First, using a pool of RNA from all tumors and two control pools of RNA from two different sets of normal tissues, the large proportion of isoforms that i) are not found in the tumor pool and/or ii) are found in one of the normal control pools, are filtered out. Second, with RNA from the individual tumors, fine-grained experiments on the small set of isoforms that remain after the first pass filtering are performed to evaluate the consistency of their tumor expression.

First pass RT-qPCR validation experiments. Because of the limitations inherent in RNA-seq data for accurate isoform identification, most isoforms predicted to be tumor-specific are expressed in some normal tissue(s). To efficiently and rapidly filter out such isoforms from further consideration, a pooled RNA screening strategy is employed. This is done with three pools of RNA—one tumor RNA pool and two control RNA pools. The tumor pool is composed of RNA from all HPV-HNSCC samples. Since the RNA expression profiles of tumors most resemble their tissue(s) of origin, the first control pool area comprises a mix of RNA from normal head and neck tissues. For those isoforms that are found in the tumor pool but not in the first control pool, a second control pool is used to evaluate their body-wide expression. The RNA for the control pools are purchased from commercial sources (such as Biochain or Origene). In the HNSCC Tumor pool, 48 HPV(-) samples are included. In the first control pool, 3 each of samples from normal larynx, pharynx, tonsil, trachea, salivary gland, esophagus, and tongue are included. In the second control pool, 3 each of samples from normal tissues from 45 different body sites are included. Those isoforms with tumor pool expression and no control pool expression are retained for subsequent experiments. Approximately 98 384-well plate RT-qPCR experiments are performed to first-pass-filter the 5,200 candidate tumor-specific isoforms using both control pools.

Second pass RT-qPCR validation experiments. To more sensitively and thoroughly evaluate the tumor-specific expression of isoforms that are not filtered out in the first pass, expression of these isoforms is measured in each of the individual 48 HPV-HNSCC tumors. No RNA sample pooling occurs in this second pass. An additional 26 384-well plate RT-qPCR experiments are performed to evaluate these isoforms, toward the goal of identifying approximately 47 RNA isoforms that are expressed in more than one tumor and not in normal tissues.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of making a synthetic polynucleotide, comprising:
selecting a cellular compartment;
identifying a first set of genetic sequences expressed in said cellular compartment;
identifying a second set of genetic sequences expressed as multiple isoforms;
generating a subset of said first and second sets of genetic sequences, said subset comprising sequences that are both (a) expressed in said cellular compartment and (b) capable of being expressed in multiple isoforms; and
generating polynucleotide primers that are complementary to one or more sequences from said subset of genetic sequences, and may further comprise sequences providing for the use of universal sequencing primers and/or unique genetic identifiers.

2. The method of claim 1 further comprising amplifying one or more genetic sequences of said subset of genetic sequences from tumor and/or normal tissue.

3. The method of claim 1, further comprising identifying the protein products of said subset of genetic sequences in tumor and/or normal tissue.

4. The method of claim 1, further comprising isolating the protein products of said subset of genetic sequences from tumor tissue.

5. The method of claim 1, wherein said polynucleotide primers further comprise additional sequences outside of the region containing the sequence of said isoforms.

6. The method of claim 1, wherein said polynucleotide primers further comprise universal sequencing sites.

7. The method of claim 1, wherein said polynucleotide primers further comprise sequences for the identification of said primers or the amplification products thereof.

8. A method of obtaining a tumor-specific biomarker, comprising:
selecting a cellular compartment;
identifying a first set of genetic sequences expressed in said cellular compartment;
identifying a second set of genetic sequences expressed as multiple isoforms; and
generating a subset of said first and second sets of genetic sequences, said subset comprising one or more genetic sequences known to be both (a) expressed in said cellular compartment and (b) capable of being expressed in multiple isoforms; and
extracting, purifying, or expressing the product of one or more genetic sequences from said subset of genetic sequences.

9. The method of claim 8 further comprising amplifying one or more genetic sequences of said subset of genetic sequences from tumor and/or normal tissue.

10. The method of claim 8, further comprising identifying the protein products of said one or more genetic sequences of said subset of genetic sequences in tumor and/or normal tissue.

11. The method of claim 8, further comprising isolating the protein products of said one or more genetic sequences of said subset of genetic sequences from tumor tissue.

12. The method of claim 8, further comprising producing the protein products of said one or more genetic sequences of said subset of genetic sequences by heterologous expression.

13. The method of claim 8, wherein said polynucleotide primers further comprise universal sequencing sites.

14. The method of claim 8, wherein said polynucleotide primers further comprise sequences for the identification of said primers or the amplification products thereof.

15. A method of generating antibodies against a tumor-specific biomarker, comprising:
selecting a cellular compartment;
identifying a first set of genetic sequences expressed in said cellular compartment;
identifying a second set of genetic sequences expressed as multiple isoforms; and
generating a subset of said first and second sets of genetic sequences, said subset comprising sequences that are both (a) expressed in said cellular compartment and (b) capable of being expressed in multiple isoforms; and
extracting, purifying, or expressing the product of one or more genetic sequences from said subset of genetic sequences; and
generating an antibody against said product.

16. The method of claim 15 further comprising amplifying said one or more genetic sequences from said subset of genetic sequences from tumor and/or normal tissue.

17. The method of claim 15, further comprising identifying the protein products of said one or more genetic sequences from said subset of genetic sequences in tumor and/or normal tissue.

18. The method of claim 15, further comprising isolating the protein products of said one or more genetic sequences from said subset of genetic sequences from tumor tissue.

19. The method of claim 15, further comprising producing the protein products of said genetic sequences by heterologous expression.

20. The method of claim 15, wherein said generating an antibody against said product further comprises immunizing an animal with said product.

21-27. (canceled)
